(19) United States
(12) Patent Application Publication
(10) Pub. No.: US 2026/0112448 A1
(43) Pub. Date: Apr. 23, 2026

(54) APPARATUS FOR GENERATING A PERSONALIZED RISK ASSESSMENT FOR NEURODEGENERATIVE DISEASE

(71) Applicant: Richard Isaacson, Boston, MA (US)

(72) Inventor: Richard Isaacson, Boston, MA (US)

(21) Appl. No.: 19/111,510

(22) PCT Filed: Sep. 13, 2023
(86) PCT No.: PCT/US2023/032582

§ 371 Date: Mar. 13, 2025

Related U.S. Application Data
(60) Provisional application No. 63/405,969, filed on Sep. 13, 2022.

(51) Int. Cl. G16B 20/20; G16B 20/10; G16B 40/20

(52) U.S. Cl. CPC G16B 20/20; G16B 20/10; G16B 40/20

(57) ABSTRACT

An apparatus for generating personalized risk assessments for neurodegenerative diseases includes a computing device that receives user data containing genetic and medical information. It processes the data to create genotype identification and gene detection modules, identifying user genotypes and relevant genetic markers. The user's mitochondrial haplogroup is examined to refine the assessment. A risk calculation module employs machine learning to weigh genetic variants against population-based data, calculating a polygenic risk score (PRS). The PRS forms a personalized risk profile, displayed through a visual interface. The disclosed systems offer a comprehensive approach to accurate risk assessment, enabling targeted interventions and informed decision-making in neurodegenerative disease management.

### CROSS-REFERENCE TO RELATED APPLICATIONS

This application claims the benefit of priority of U.S. Provisional Application Ser. No. 63/405,969, filed on Sep. 13, 2022, and entitled “ApoGenesis: Driving the next generation of precision medicine for neurological diseases,” which is incorporated by reference herein in its entirety.

### FIELD OF THE INVENTION

The present invention generally relates to the field of bioinformatics and genetic data analysis. In particular, the present invention is directed to an apparatus for generating a personalized risk assessment for neurodegenerative disease.

### BACKGROUND

Neurodegenerative diseases, such as Alzheimer's disease and related conditions, represent a substantial burden on healthcare systems worldwide due to their intricate interplay of genetic and environmental influences. These diseases are characterized by the gradual degeneration of neurons, resulting in cognitive decline, impaired motor functions, and diminished quality of life for affected individuals. As the global population ages, the prevalence of neurodegenerative diseases continues to rise, highlighting the urgency to develop more effective strategies for prevention, early detection, and intervention. Traditional risk assessment methods often provide a broad perspective on disease susceptibility, potentially overlooking the nuanced interactions between an individual's unique genetic composition, lifestyle choices, and environmental factors. This limitation can hinder the accuracy of risk predictions and hinder the ability to provide targeted interventions to those most in need.

### SUMMARY OF THE DISCLOSURE

In an aspect, an apparatus for generating a personalized risk assessment for neurodegenerative disease includes a computing device configured to receive user data pertaining to a user, wherein the user data comprises user genetic data. The apparatus is configured to process the user genetic data, wherein process the user genetic data of the user, wherein processing the user genetic data comprises generating a genotype identification module using the user genetic data, wherein the genotype identification module is configured to identify a user genotype. Processing the user genetic data further generate a gene detection module using the user genetic data, wherein the gene detection module is configured to determine the presence of one copy of an apolipoprotein (ApoE)4 gene and identify the upstream and downstream regulatory region of ApoE4 and translocase of outer mitochondrial membrane 40 (TOMM40) gene to assess potential impact on disease risk. Last, processing the user genetic data comprises examining the user's mitochondrial haplogroup. The apparatus is further configured to generate a genetic profile, wherein the genetic profile comprises a personalized risk profile. Finally, the apparatus display the personalized risk profile through a display device.

In another aspect, a method for generating a personalized risk assessment for neurodegenerative disease includes receiving, by at least a processor, user data, wherein the user data comprises a user's genetic data. The method includes identifying, by at least a gene detection module of the at least a processor, wherein the at least a genotype identification module is configured to identify a user genotype. The method includes determining, by at least a gene detection module of the at least a processor, wherein the at least a gene detection module comprises identifying the presence of one copy of the ApoE4 variant. The method includes processing, by at least an epigenetic analysis module, wherein the at least a epigenetic analysis module is configured to process a genome. The method further includes calculating, using at least a risk calculation module, a polygenic risk score (PRS).

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an exemplary system for an apparatus for generating a personalized risk assessment for neurodegenerative disease;

FIG. 2 is a block diagram of an exemplary machine-learning process;

FIG. 3 is a diagram of an exemplary embodiment of neural network;

FIG. 4 is a diagram of an exemplary embodiment of a node of a neural network;

FIG. 5 is a flow diagram illustrating an exemplary workflow of a method for generating a personalized risk assessment for neurodegenerative disease; and

FIG. 6 is a block diagram of a computing system that can be used to implement any one or more of the methodologies disclosed herein and any one or more portions thereof.

The drawings are not necessarily to scale and may be illustrated by phantom lines, diagrammatic representations, and fragmentary views. In certain instances, details that are not necessary for an understanding of the embodiments or that render other details difficult to perceive may have been omitted. Like reference symbols in the various drawings indicate like elements.

### DETAILED DESCRIPTION

At a high level, aspects of the present disclosure are directed to systems and methods for generating a personalized risk assessments for a neurodegenerative disease.

Aspects of the present disclosure can be used to address various aspects, including early detection and proactive management of disease risk, where timely intervention can lead to better outcomes. Aspects of the present disclosure can also be used to optimize treatment strategies and tailor medical interventions for improved user care. This is so, at least in part, because of the integration of advanced computational algorithms that analyze complex genetic and biological data to provide actionable insights. A key advantage of the present disclosure is the capability it affords for a personalized risk assessment that combines genetic information, traditional biological data, and specific genetic risk factors. By amalgamating these diverse sources of data, the disclosed systems and methods enable a more holistic assessment of an individual's vulnerability to neurodegenerative diseases, leading to more informed healthcare decisions.

Aspects of the present disclosure allow for an understanding of an individual's susceptibility to neurodegenerative diseases, ultimately enhancing the accuracy of risk predictions and enabling targeted interventions. Exemplary embodiments illustrating aspects of the present disclosure are described below in the context of several specific examples.

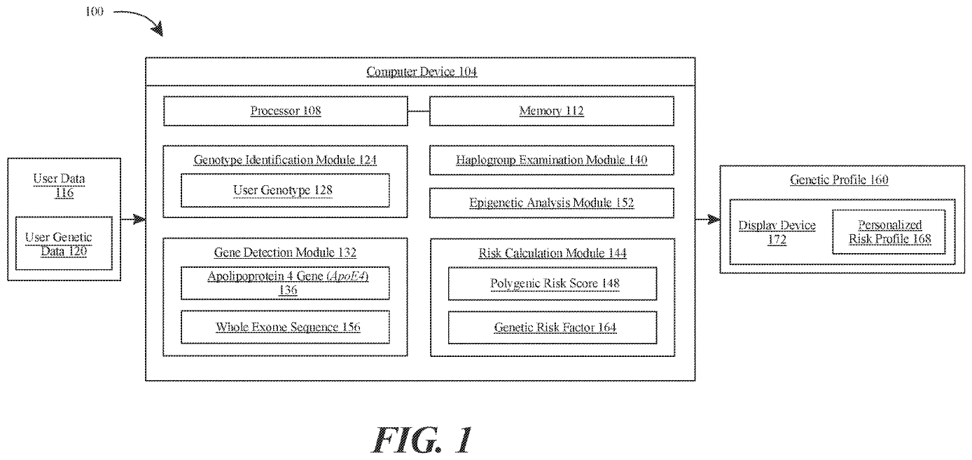

Referring now to FIG. 1, an exemplary embodiment of an apparatus 100 for generating a personalized risk assessment for neurodegenerative disease is illustrated. System includes a computing device. Computing device may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device may include a single computing device operating independently, or may include two or more computing device operating in concert, in parallel, sequentially or the like; two or more computing devices may be included together in a single computing device or in two or more computing devices. Computing device may interface or communicate with one or more additional devices as described below in further detail via a network interface device. Network interface device may be utilized for connecting computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device may include but is not limited to, for example, a computing device or cluster of computing devices in a first location and a second computing device or cluster of computing devices in a second location. Computing device may include one or more computing devices dedicated to data storage, security, distribution of traffic for load balancing, and the like. Computing device may distribute one or more computing tasks as described below across a plurality of computing devices of computing device, which may operate in parallel, in series, redundantly, or in any other manner used for distribution of tasks or memory between computing devices. Computing device may be implemented using a “shared nothing” architecture in which data is cached at the worker, in an embodiment, this may enable scalability of system 100 and/or computing device.

With continued reference to FIG. 1, computing device may be designed and/or configured to perform any method, method step, or sequence of method steps in any embodiment described in this disclosure, in any order and with any degree of repetition. For instance, computing device may be configured to perform a single step or sequence repeatedly until a desired or commanded outcome is achieved: repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, apparatus 100 may include a memory 112. Memory 112 may contain instructions configuring processor 108 to perform actions consistent with this disclosure. Memory 112 is communicatively connected to processor 108. As used in this disclosure, “communicatively connected” means connected by way of a connection, attachment or linkage between two or more relata which allows for reception and/or transmittance information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure. As used in this disclosure, “communicatively connected” means connected by way of a connection, attachment or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference to FIG. 1, processor 108 may perform determinations, classification, and/or analysis steps, method, processes, or the like as described in this disclosure using machine-learning processes. A “machine-learning process,” as used in this disclosure, is a process that automatedly uses a body of data known as “training data” and/or a “training set” (described further below in this disclosure) to generate an algorithm that will be performed by a processor 108/module to produce outputs given data provided as inputs; this is in contrast to a non-machine learning software program where the commands to be executed are determined in advance by a user and written in a programming language. Machine-learning process may utilize supervised, unsupervised, lazy-learning processes and/or neural networks, described further below.

With continued reference to FIG. 1, computing device 104 may receive user data 116 pertaining to a user, wherein the user data 116 comprises user genetic data 120. As used in this disclosure, “user data” refers to information related to a user. In some cases, user may include, without limitation, an individual, a group of individual, a small business, a clinic, any medical entity and/or the like. In an embodiment, user data may include user input data. In an embodiment, receiving user data may further comprise at-home blood test data. In a non-limiting example, the at-home blood test data may encompass measurements of relevant biomarkers, including but not limited to amyloid levels and other indicators associated with neurodegenerative disease risk (e.g. total cholesterol, triglycerides, desmosterol, fibrinogen, LDL-C, and the like). Users may be provided with an at-home blood test kit. As used in this disclosure, “at-home blood test kit” refers to a user-friendly at home test that may screen for diabetes, hormone imbalances, and heart health. As a non-limiting example, the at-home test may comprise instructions and materials for collecting blood samples in a minimally invasive manner, such as a finger prick test. In another embodiment, user data 116 may further comprise direct-to-consumer (DTC) genetic data. In a non-limiting example, DTC testing may generate DTC genetic data. In DTC testing, consumers collect a DNA sample at home, such as saliva or a cheek swab, and send it to a private laboratory for testing. These companies may provide information about genetic traits, health risks, and ancestry. In a further embodiment, user data 116 may further comprise traditional biological data. As used in this disclosure, “traditional biological data” refers to a health-related information that may be associated with user care or clinical trials. In some cases, traditional biological data may include but not limited to user's history, clinical findings, diagnostic test results, and the like. In a non-limiting example, traditional biological data may encompass a comprehensive array of healthcare-related information, including but not limited to medical history, diagnostic reports, treatment records, physical examination findings, vital signs measurements, healthcare visits, clinical notes, family medical history, and demographic details. The integration of traditional biological data with user genetic data 120 and additional at-home blood test data may enhance the depth and accuracy of the generated genetic profile and personalized risk assessment. In a further embodiment, receiving user genetic data 120 and at-home blood test data may be obtained using the at-home kit, apparatus 100 proceeds to process these data sets using a combination of computational algorithms and analytical modules. By integrating genetic information with clinical data, apparatus generates a comprehensive genetic profile that encompasses a personalized risk assessment for neurodegenerative diseases, such as Alzheimer's disease.

Still referring to FIG. 1, process user genetic data 120 of the user, wherein processing the user genetic data comprises generating a genotype identification module 124 using user genetic data 120, wherein the genotype identification module 124 is configured to identify a user genotype 128. As used in this disclosure, a “user genotype” refers to the specific genetic composition and variations present within the user's genome, contributing to their unique genetic profile and potential disease predispositions. In an embodiment, genotype identification module 124 is configured to identify user's ApoE genotype. As described in this disclosure, “ApoE genotype” refers to the specific genetic variants present within the apolipoprotein E gene (ApoE gene) 136, a key genetic determinant that has been extensively associated with varying degrees of susceptibility to neurodegenerative diseases. As a non-limiting example, a user's genetic data may be analyzed using the genotype identification module. Upon processing the genetic information, the genotype identification module identifies specific variations within the ApoE gene 136. Analysis may begin with the input of user genetic data, which is often represented as sequences of DNA. Genotype identification module may be programed to locate and identify predetermined genetic markers of interest within this data. For example, in the case of ApoE genotype identification, genotype identification module may search for variations within the ApoE gene 136. A “variant calling” process, as used in this disclosure, refers to identification of genetic variation, or alleles, at specific positions in the DNA sequence. For example, ApoE genotype determination, it may seek out the presence of alleles ε2, ε3, or ε4, at designated positions within the ApoE gene 136. Based on these identified variations, genotype identification module may determine the user's genotype. If, for instance, the user's genetic data holds one ε3 allele and one ε4 allele, genotype identification module may classify their ApoE genotype as ε3/ε4. Subsequently, genotype identification module may generate a report or output that outlines the identified alleles, the resultant genotype, and potentially relevant information such as associated disease risks. Depending on the context, the identified genotype might be further cross-referenced against established databases or reference populations to potential disease associations or levels of risk. In a further embodiment, processing the genome comprises conducting whole genome sequencing (WGS) 156. As used in this disclosure, “genome” refers to the complete set of genetic material or DNA present within an organism's cells, encompassing all the genes, non-coding regions, and regulatory elements that may play a role in the organism's genetic makeup. In an embodiment, conducting WGS 156 may involve deciphering the sequence of nucleotides (adenine, cytosine, guanine, and thymine) that constitute an individual's DNA. The sequencing process may provide an overview of the genetic variations, mutations, and markers that contribute to an individual's unique genetic profile. Determining a whole exome sequence may involve selectively sequencing only the protein-coding regions of an user genome, known as exome. Exome may comprise a small fraction of the entire genome but is of significant interest as it may encode proteins critical for various cellular functions. Whole exome sequencing may be performed using targeted capture techniques that may isolate and sequence the exonic portions of DNA. Obtained sequencing data may then be analyzed to identify variations, mutations, and genetic markers that may contribute to an user risk of neurodegenerative diseases. For instance, it may enable the identification of disease-associated variants, the determination of genetic predispositions, and the exploration of personalized healthcare strategies. In a non-limiting example, the results of WGS may unveil a user's genetic susceptibility to neurodegenerative diseases, enhancing the accuracy of the personalized risk assessment and guiding the formulation of targeted clinical management strategies. In another embodiment, processing the genome may involve the determination of a WGS 156. This may intricate genetic analysis methodology delves into the complete sequence of nucleotides-adenine, cytosine, guanine, and thymine—that constitute an individual's DNA. The determination of WGS 156 encompasses decoding of the organism's entire genetic makeup, encompassing genes, non-coding regions, and regulatory elements. Determination may yield a comprehensive genetic blueprint, unraveling genetic variations, mutations, and markers unique to the individual.

With continued reference to FIG. 1, computer device 104 may include a gene detection module 132, wherein processing the user genetic data 120 comprises generating a gene detection module 132 using the user genetic data 120. As used in this disclosure, a “gene detection module” refers to a computational entity designed to identify and analyze specific genetic markers, variants, or sequences within the user's genetic data. Gene detection module may pinpoint the presence or absence of distinct genetic attributes, including but not limited to genes associated with disease susceptibility, biomarkers, and other genetic indicators. In an embodiment, gene detection module may determine the presence of one copy of an apolipoprotein (ApoE) 4 gene. As used in this disclosure, an “apolipoprotein 4 gene” refers to a specific genetic locus encoding the ApoE4 protein variant. ApoE4 may be associated with distinctive structural and functional attributes within the apolipoprotein E family, potentially contributing to altered lipid metabolism and influencing an individual's susceptibility to certain health conditions, including neurodegenerative diseases. In a non-limiting example, gene detection module may utilize computational algorithms to scrutinize user genetic data with a high degree of precision. Upon receiving user genetic data, gene detection module may employ bioinformatics algorithms to scan the DNA sequences for specific genetic markers, variants, or patterns associated with the target genes, such as ApoE4. The algorithms within gene detection module may be designed to recognize unique sequences, mutations, or structural characteristics that correspond to ApoE4. Gene detection module may encompass methods like sequence alignment, pattern matching, and statistical modeling to distinguish relevant genetic patterns from noise and variations inherent to the genetic code. Gene detection module may then accurately pinpoint the presence of the ApoE4 gene variant with user genetic data. In another embodiment, risk assessment is based on genetic factors associated with the ApoE gene. As used in this disclosure, “risk assessment” refers to the systematic evaluation of an individual's potential vulnerability to specific health conditions, particularly neurodegenerative diseases, based on their genetic makeup. Genetic factors encompassing ApoE gene may be examined to ascertain potential impact on disease risk. By considering variations and mutations within the ApoE gene, risk assessment may discern patterns associated with increased or decreased susceptibility to neurodegenerative diseases. The risk assessment process may involve quantifying the influence of these genetic factors and integrating them into a risk assessment profile for the individual. As a non-limiting example, insights may be derived from ApoE and associated genetic factors. By factoring these, risk assessment process may enhance the precision of personalized healthcare strategies and facilitates targeted clinical decision-making. In a further embodiment, wherein generating gene detection module 132 further comprises identifying the upstream and downstream regulatory region of ApoE and TOMM40. As used in this disclosure, “upstream and downstream” refers to distinct genetic regions that are positioned before (upstream) or after (downstream) a particular gene locus along the DNA strand. “Upstream”, as described in this disclosure, indicates the genetic region preceding the gene locus, often containing regulatory elements that may influence gene expression and activity. Conversely, “downstream” refers to the genetic region succeeding the gene locus, which may also contain regulatory elements affecting gene function. The identification of these regulatory regions within the Apo and TOMM40 genes is essential for understanding how these genes are controlled and regulated in the context of genetic data analysis. As used in this disclosure, “TOMM40” gene refers to a specific genetic locus that encodes the Translocase of Outer Mitochondrial Membrane 40 (TOMM40) protein. The TOMM40 gene may be situated within the human genome and facilitating the transport of proteins into mitochondria, the cellular organelles responsible for energy production. As a non-limiting example, gene detection module may be designed and configured to perform an analysis of the genetic information associated with the ApoE and TOMM40 genes. Gene detection module may leverage known genetic markers and regulatory motifs to precisely delineate the boundaries of the upstream and downstream regulatory regions. To identify the upstream region, gene detection module may scan the genetic sequence upstream of the ApoE and TOMM40 genes, searching for distinctive regulatory elements that are known to influence gene expression and activity. These regulatory elements might include promoter regions, enhancers, and transcription factor binding sites. Gene detection module algorithms may analyze the sequence patterns and structural characteristics that signify the presence of these regulatory elements, effectively demarcating the boundary of the upstream regulatory region. Similarly, for the downstream region, gene detection module may extend its analysis to the genetic sequence downstream of ApoE and TOMM40 genes. It may recognize genetic motifs that are indicative of post-transcriptional regulatory elements, mRNA stability elements, and other components that contribute to gene regulation. By systematically assessing the sequence composition and arrangement, gene detection module may define the extent of the downstream regulatory region.

With continued reference to FIG. 1, gene detection module 132 may be configured to process user genetic data 120. A haplogroup examination module 140 may be configured to examine user mitochondrial haplogroup using user genetic data 120. As used in this disclosure, “mitochondrial haplogroup” signifies a specific genetic cluster characterized by a set of variations within the mitochondrial DNA that can trace ancestral origins and potential associations with specific health traits. In a non-limiting example, as part of a detailed analysis, participants that enroll in the Comparative Effectiveness Alzheimer's & Dementia Registry (CEDAR) may be considered, including those who underwent direct-to-consumer (DTC) genetic testing along with their family members. The approach in this example may involve applying a standard CEDAR risk assessment. which may include a evaluation of relevant risk factors. To compute and generate an Alzheimer's disease (AD) polygenic risk score (PRS), In this process, a standard CEDAR risk assessment is applied to user data. Additionally, a customized PRS specific to AD may be calculated. Calculation may take into account various genetic markers and factors associated with AD risk. PRS may be determined using a combination of genetic data analysis, machine learning models, and relevant genetic risk factors The PRS may then qualitatively modified by considering mitochondrial DNA (mtDNA) haplogroup status, genetic variants impacting APOE alleles, and single nucleotide polymorphisms (SNPs) linked to medical conditions affecting AD risk. This approach may enable an assessment of how genomic data influences risk profiles and clinical care. In an embodiment, haplogroup examination module 140 further comprises analyzing specific genetic markers associated with mitochondrial DNA. As used in this disclosure, “haplogroup examination module” refers to a machine-learning component of apparatus 100 designed to perform targeted investigation into an individual's mitochondrial DNA, particularly focusing on haplogroups, specific genetic lineages or clusters that share characteristic variation. Haplogroup examination module may receive input data, including the user's mitochondrial DNA sequence and relevant genetic markers. The module may process input and generate output that identify the specific haplogroup to which the user's mitochondrial DNA belongs. For example, a dataset of mitochondrial DNA sequences with known haplogroup assignments may be used to train haplogroup examination module. Training data may enable the module to learn the genetic patterns and variations associated with different haplogroups, enhancing its accuracy in identifying haplogroups in user data. Machine learning may be employed in this example, haplogroup examination module may enhance the ability to provide information about individual's ancestral lineage and genetic heritage. As a non-limiting example, a more detailed examination of mitochondrial genetic markers associated with haplogroups, to uncover genetic distinctions may be entailed. This analysis may involve the evaluation of specific genetic variations that may indicate particular haplogroups and contribute to an individual's mitochondrial genetic profile. In another embodiment, wherein processing the user genetic data 120 further comprises determining the ancestral lineage and genetic characteristics of the user's mitochondrial genome. As used in this disclosure, “ancestral lineage” refers to the genetic heritage and lineage tracing that unveils an individual's genetic origins and ancestral heritage. Determining the ancestral lineage of the user's mitochondrial genome may involve an in-depth investigation into the user's mitochondrial genome to extract not only the ancestral lineage information but also the distinctive genetic characteristics that define their mitochondrial makeup. By analyzing specific mitochondrial genetic markers associated with various populations and geographic regions, apparatus may aim to unveil the historical migratory paths and genetic makeup of an individual's maternal lineage. As used in this disclosure, “genetic characteristic” refers to specific features and traits encoded within an individual's genetic material, encompassing variations, mutations, and sequences that collectively contribute to their genetic makeup. These genetic characteristics serve as the foundation for understanding an individual's predisposition to specific traits, health conditions, and potential susceptibility to various diseases. As a non-limiting example, apparatus may employ a comprehensive analysis of specific genetic markers associated with mitochondrial DNA, which may have been linked to different populations and geographic regions. This analysis may involve comparing the user's mitochondrial DNA markers with a reference database containing genetic data from diverse populations. By identifying genetic variations specific to certain ancestral groups, the apparatus can infer the user's ancestral lineage and geographic origins. Furthermore, the analysis of genetic characteristics within the user's mitochondrial genome markers and variations that may be encoded in mitochondrial DNA. This process may involve scrutinizing the sequence of nucleotides within the mitochondrial genome to identify distinct genetic patterns and variations unique to the individual's genetic makeup. By comparing these variations against established databases and known genetic markers associated with certain traits or conditions, the analysis may aim to uncover specific genetic characteristics that contribute to an individual's risk profile for neurodegenerative diseases. This may involve examining variations in mitochondrial genes that are known to influence health traits and disease susceptibility. By comparing the user's mitochondrial genetic characteristics to known patterns and associations, apparatus may provide insights into the potential health implications and disease risks associated with their mitochondrial genetic makeup. As another non-limiting example, a user's mitochondrial genome analysis may reveal genetic characteristics associated with increased or decreased susceptibility to specific diseases, metabolic traits, or other health conditions. This information may contribute to a comprehensive understanding of the user's genetic predispositions and provides valuable insights for personalized risk assessment and clinical management strategies.

Still referring to FIG. 1, apparatus 100 may further be configured to generate a genetic profile 160. As used in this disclosure, a “genetic profile” refers to a comprehensive compilation of an individual's genetic information, encompassing various genetic markers, variations, mutations, and sequences that collectively depict their genetic makeup. The genetic profile 160 may provide a view of an individual's genetic characteristics and serves as a foundation for assessing potential health risks, traits, and susceptibilities. As a non-limiting example, genetic profile may be created through the integration of data obtained from various genetic analysis modules, including genotype identification module 124 and gene detection module 132. These modules collectively decipher an individual's genetic code, identifying specific genes, variants, and markers associated with various health conditions and traits. Genetic profile captures both attributes and commonalities present within an individual's genetic material, contributing to a comprehensive understanding of their genetic predispositions. Genetic profile may encompass a wide range of genetic information, such as the presence of specific alleles, mutations linked to disease risk, and markers associated with metabolic traits and health conditions. Those data may be collated and organized into a structured format that aids in risk assessment, clinical decision-making, and personalized healthcare strategies. As another non-limiting example, genetic profile may include details about the user's ApoE genotype, the presence of specific risk alleles, mitochondrial haplogroup, and other genetic factors that influence disease susceptibility. By analyzing the genetic profile, healthcare professionals can tailor clinical management plans to address individualized risk factors, provide targeted interventions, and promote proactive health management strategies. In an embodiment, wherein the genetic profile comprises a personal risk profile.

With continued reference to FIG. 1, With continued reference to FIG. 1, genetic profile 160 may comprise a personalized risk profile 168. As used in this disclosure, a “personalized risk profile” refers to a customized assessment that integrates an individual's genetic information with other relevant factors, such as family history, lifestyle, and clinical data, to determine susceptibility to specific health conditions, particularly neurodegenerative diseases. The personalized risk profile 168 quantifies an individual's likelihood of developing certain diseases based on a combination of genetic predispositions and environmental factors. As a non-limiting example, personalized risk profile 168 may also involve the aggregation and analysis of data from various genetic analysis modules, including the genotype identification module 124 and the gene detection module 132. These modules contribute essential genetic insights that inform the risk assessment process. Additionally, risk calculation module 144 factors in traditional biological data and other pertinent information to refine the risk estimation. Personalized risk profile 168 may provide a comprehensive overview of an individual's health risks, highlighting areas of concern and potential vulnerabilities. It may serve as a valuable tool for healthcare practitioners to tailor preventive strategies, recommend targeted interventions, and guide clinical decisions. By considering an individual's genetic makeup in conjunction with other relevant factors, personalized risk profile may enhance disease prediction accuracy and facilitates proactive healthcare management. As a non-limiting example, personalized risk profile may indicate an individual's increased susceptibility to neurodegenerative diseases based on the presence of certain genetic markers, such as the ApoE4 allele, in combination with lifestyle factors and other clinical indicators.

With continued reference to FIG. 1, apparatus 100 may further comprise a risk calculation module 144. As used in this disclosure, a “risk calculation module” refers to a machine-learning component configured to quantitatively evaluate an user's risk for developing specific health conditions based on a multifaceted analysis of various data input. The risk calculation module may receive inputs such as, but not limited to, user genetic data, medical history, lifestyle information, and potentially other relevant parameters. Leveraging machine-learning algorithms, risk calculation module may process these inputs and generates outputs that include a personalized risk assessment profile for specific health conditions, highlighting the likelihood of their occurrence over time. Training data for risk calculation module could encompass a diverse range of datasets, including, but not limited to, anonymized medical records, genetic databases, lifestyle data, and historical disease incidence rates. Training data may include sets of user data and genetic information correlated to risk profiles. By utilizing this data, the machine-learning model may learn complex patterns, correlations, and interactions that contribute to disease susceptibility. Potential training data sources may include large-scale epidemiological studies, genomic databases, electronic health records, and longitudinal health surveys. Risk calculation module may learn to recognize patterns and associations between input variables and disease outcomes during training process. Training process may involve adjusting model parameters to minimize prediction errors and improve the accuracy of risk assessments. By learning from historical data, the machine-learning model may apply its acquired knowledge to new data and generate personalized risk assessments for individuals, enabling informed decision-making and personalized healthcare recommendations. In some embodiments, risk calculation module particularly neurodegenerative diseases, based on a comprehensive analysis of their genetic information and relevant clinical data. The risk calculation module 144 may perform algorithms that combine various genetic markers, genetic characteristics, and other pertinent factors to generate a quantifiable risk score. This score may represent the individual's estimated probability of encountering certain health conditions within a defined timeframe. The risk calculation process may take into account the interactions between different genetic variants, the influence of environmental factors, and the impact of genetic characteristics on disease susceptibility. One significant aspect of risk calculation module 144 is its utilization of advanced methodologies to analyze specific attributes of DNA and identify distinctive features within the genetic material. Risk calculation module 144 may integrate the outcomes of the genotype identification module 124, the gene detection module 132, and other relevant genetic insights to enhance the accuracy of risk assessment. Risk calculation module 144 may consider a wide array of genetic and non-genetic factors (gene interactions, lifestyle choices, medical history, demographic data, and the like). By incorporating these multifaceted aspects, risk calculation module 144 may generate a risk profile that empowers healthcare practitioners and individuals to make informed decisions regarding disease prevention, management, and personalized treatment strategies. As a non-limiting example, the risk calculation module may integrate genetic markers associated with the ApoE gene, mitochondrial haplogroup status, and relevant SNPs into a sophisticated algorithm. This algorithm calculates an individual's polygenic risk score (PRS), offering insights into their predisposition to neurodegenerative diseases. The resulting risk score informs healthcare professionals about the individual's likelihood of disease onset, enabling them to develop tailored intervention plans and monitor disease progression effectively. In an embodiment, the risk calculation module may be configured to identify the user's risk for the onset of neurodegenerative diseases based on a comprehensive analysis that incorporates genomic information, traditional biological data, and specific genetic risk factors. This multifaceted analysis involves an intricate assessment of an individual's genetic makeup, considering variations, mutations, and sequences that collectively contribute to their genetic characteristics. Risk calculation module may combine these genetic attributes with relevant clinical information, such as medical history, lifestyle factors, and demographic data. Both genomic and traditional biological data may be integrated, risk calculation module may generate risk assessment profile that provides an overview of an individual's potential vulnerability to neurodegenerative diseases. Specific genetic risk factors associated with the individual's genetic markers, including the ApoE gene and mitochondrial haplogroup status, may be examined and incorporated into the risk assessment algorithm. Risk calculation module may employ advanced computational techniques to evaluate the interactions between genetic factors and their impact on disease susceptibility. It may quantify the cumulative effect of these factors and generates a risk score that quantifies the likelihood of disease onset within a defined timeframe. Personalized risk assessment may equip healthcare professionals with valuable insights to develop tailored prevention and intervention strategies, enabling proactive management of neurodegenerative diseases. As a non-limiting example, risk calculation module may consider the individual's ApoE genotype, mitochondrial haplogroup, and specific genetic markers associated with neurodegenerative diseases. These factors, in conjunction with other clinical information, contribute to a comprehensive risk assessment profile that aids healthcare practitioners in making informed decisions for disease management and personalized care. In another embodiment, the neurodegenerative disease further comprises Alzheimer's disease (AD). In a further embodiment, the analysis of genetic risk factors 164 takes into account an extensive range of risk factors associated not only with AD but also with other neurodegenerative diseases, cardiovascular disease, inflammatory disease, and metabolism-related conditions, and the like. The analysis process may involve an examination of an individuals genetic data to identify specific variations, mutations, and sequences that may contribute to their susceptibility to various health conditions. Genetic markers linked to AD risk, such as the ApoE gene and other relevant genetic loci, may be scrutinized to ascertain their potential impact on disease predisposition. Additionally, genetic factors relevant to cardiovascular health, inflammatory response, and metabolic function may also be assessed to create a comprehensive overview of the individual's genetic predispositions. The risk assessment conducted by apparatus 100 may employ algorithms that integrate the collective influence of these genetic factors on the overall disease risk. By examining a broad spectrum of risk factors across different health domains, apparatus may generate a more holistic risk assessment profile. “Risk assessment profile” refers to a detailed overview of an individual's potential vulnerability to various health conditions. Risk assessment profile may take into account genetic, lifestyle, and medical factors that collectively contribute to their overall health risk. This profile may offer a holistic view of an user's health outlook, aiding in the identification of potential risk areas and enabling personalized recommendations for disease prevention and management strategies. This profile may assist healthcare professionals in understanding an individual's multifaceted susceptibility to not only AD but also other related health conditions. As a non-limiting example, the apparatus may consider genetic markers associated with lipid metabolism for cardiovascular disease, markers related to immune response for inflammatory diseases, and markers associated with metabolic pathways for metabolism-related conditions. The integration of these diverse genetic factors into the risk assessment process enhances the accuracy and clinical relevance of the personalized risk profile, empowering healthcare practitioners to formulate targeted interventions and comprehensive disease management strategies. Additionally, in another embodiment, wherein the risk calculation module is configured to integrate the reports effected size of individual single nucleotide polymorphisms (SNPs) into the risk assessment process. As used in this disclosure, “single nucleotide polymorphisms” (SNPs) refers to small variations at specific positions in an individual's DNA sequence where a single nucleotide (A, T, C, or G) differs between individuals or populations. The analysis process may involve scrutinizing specific SNPs that have been associated with disease onset, progression, or susceptibility. Specific analysis step may take place within risk calculation module, where the processor may focus on evaluating these genetic markers to gain insights into an individual's propensity for different health conditions. Processor may retrieve information about SNPs from various sources, such as publicly available genomic databases, scientific literature, and curated datasets. These repositories may store extensive information about genetic variations and their associations with disease. The processor may use this information to identify relevant SNPs and reported associations with specific health conditions. By scrutinizing these SNPs, the algorithm may effectively discern patterns that contribute to an individual's genetic predisposition to neurodegenerative diseases and other health-related outcomes. These SNPs may act as genetic markers, providing insights into an individual's predisposition to various health conditions. Effect sizes of SNPs may be reported, indicate the magnitude of influence each genetic variation may exert on disease risk. Risk calculation module may utilize the effect sizes to weigh the contribution of each SNP in the context of an individual's genetic makeup. Effect sizes may be integrated into risk assessment algorithm, the module may generate a precise risk profile. As a non-limiting example, risk calculation module may consider SNPs associated with Alzheimer's disease, cardiovascular conditions, metabolic health, and other relevant health factors. The integration of effect sizes allows for the prioritization of influential SNPs, contributing to a more accurate estimation of an individual's overall disease susceptibility. By considering both the genetic makeup and the magnitude of impact of specific SNPs, risk calculation module may enhance the reliability of the personalized risk assessment. In a further embodiment, wherein risk calculation module may be configured to weigh the impacts of individual variants against population-based incident reports. Within this context, the term “population-based incident reports” refers to statistical data that outlines the occurrence and prevalence of specific health conditions, particularly in reference to a given population or demographic group. Risk calculation module may factor in both the genetic variants present in an individual's genome and the broader context of disease prevalence within the population. Integration of information may help to contextualize an individual's genetic risk in relation to the general occurrence of the disease. Risk calculation module may assign weight to each genetic variant based on its reported impact and prevalence within the population. By comparing an individual's genetic makeup to the population-based incident reports, risk calculation module may enhance the accuracy of risk assessment. Variants that are less common in the population but have a substantial impact on disease risk for an individual can be appropriately prioritized in the risk assessment process. Conversely, variants that are prevalent in the population but have minimal impact on disease risk for an individual may receive lower weight. An additional step within risk calculation module may be entailed, where the algorithm may evaluate the reported effect sizes and prevalence of genetic variants across diverse populations. The processor may assess relative frequency of these variants within different populations and references databases that provide information on their prevalence. By doing so, the algorithm may identify variants that are common but have limited individual-level impact on disease risk. In this way, risk calculation module may take into account not only the effect sizes of genetic markers but also their prevalence within the population. Apparatus may allow risk calculation module to generate a risk profile considers the genetic predisposition of the individual and places it within the broader context of disease prevalence. As a result, the personalized risk assessment becomes more refined and tailored, providing a more realistic estimation of an individual's vulnerability to specific health conditions. Risk calculation module may be configured to weigh the impacts of individual variants against population-based incident reports may support healthcare practitioners in making well-informed decisions and assisting individuals in adopting suitable disease prevention and management strategies. As a non-limiting example, risk calculation module may analyze genetic variants associated with Alzheimer's disease and compare their impact against population-based incident reports to derive a more accurate risk assessment for an individual's susceptibility to the disease.

With continued reference to FIG. 1, risk calculation module 144 may further be configured to aggregate data from various genetic markers, including genotypes of significant genetic risk factors such as the APOE gene alleles. This integration of genetic marker data contributes to the overall risk assessment process by incorporating multiple indicators that collectively influence an individual's predisposition to neurodegenerative diseases and related conditions. As used in this disclosure, “genetic markers” refers to specific points within an individual's genetic material that are associated with particular traits, characteristics, or diseases. Risk calculation module may leverage data from genetic markers to assemble a comprehensive view of an individual's genetic landscape, particularly in relation to the risk of developing neurodegenerative diseases. The inclusion of genetic markers such as ApoE gene alleles may provide risk calculation module with nuanced insights into an individual's genetic susceptibility to neurodegenerative diseases. By considering multiple markers in tandem, risk calculation module may generate a risk assessment that reflects the complex interplay of genetic factors contributing to disease vulnerability. For example, healthcare practitioners may access a comprehensive understanding of an individual's risk profile, enabling them to offer personalized preventive measures and tailored clinical management strategies. As a non-limiting example, risk calculation module may incorporate genotypic data from the ApoE gene alleles, along with data from other relevant genetic markers associated with neurodegenerative diseases.

With continued reference to FIG. 1, risk calculation module 144 may be configured to calculate a polygenic risk score (PRS) 148. As used in this disclosure, a “polygenic risk score” (PRS) refers to a numerical representation of an individual's genetic predisposition to developing neurodegenerative diseases, incorporating data from multiple genetic markers and their associated impact on disease risk. PRS may quantify the cumulative effect of various genetic factors on an individual's susceptibility to neurodegenerative diseases, providing a quantitative measure of risk level. This score may amalgamate the individual's genetic makeup, including alleles, mutations, and variations associated with relevant genes. By integrating this diverse genetic information, risk calculation module may generate PRS that reflects the individual's overall genetic risk profile. PRS may be computed based on an algorithm that takes into account the reported effect sizes of specific genetic markers, along with their respective contributions to disease risk. The algorithm may assign weights to these markers based on their significance and impact, ultimately generating a composite score that signifies the individual's predisposition to neurodegenerative diseases. The determination of these weights may involve a comprehensive analysis that may consider various factors, including the existing body of scientific literature, epidemiological studies, and large-scale genetic database. The processor may obtain these weights from established research findings and studies that have investigated the relationship between specific genetic markers and their association with disease risk. These weights may be derived from statistical analysis that quantify the strength of the relationship between each genetic marker. In some embodiments, processor 108 may use a weight determination machine-learning module to generate the marker weights. Weight determination machine-learning module may receive as input a genetic marker and output a weight. Training data for weight determination machine-learning model may include a plurality of genetic markers correlated to associated weights. In some embodiments, processor 108 may look up a genetic marker in a weight look up table to find the associated weight. In some embodiments, weights may be determined using a language processing module. Language processing module may use a variety of input data, such as the existing body of scientific literature, epidemiological studies, and large-scale genetic database to determine weights. Language processing module may include any hardware and/or software module. Language processing module may be configured to extract, from the one or more documents, one or more words. One or more words may include, without limitation, strings of one or more characters, including without limitation any sequence or sequences of letters, numbers, punctuation, diacritic marks, engineering symbols, geometric dimensioning and tolerancing (GD&T) symbols, chemical symbols and formulas, spaces, whitespace, and other symbols, including any symbols usable as textual data as described above. Textual data may be parsed into tokens, which may include a simple word (sequence of letters separated by whitespace) or more generally a sequence of characters as described previously. The term “token,” as used herein, refers to any smaller, individual groupings of text from a larger source of text; tokens may be broken up by word, pair of words, sentence, or other delimitation. These tokens may in turn be parsed in various ways. Textual data may be parsed into words or sequences of words, which may be considered words as well. Textual data may be parsed into “n-grams”, where all sequences of n consecutive characters are considered. Any or all possible sequences of tokens or words may be stored as “chains”, for example for use as a Markov chain or Hidden Markov Model.

Still referring to FIG. 1, language processing module may operate to produce a language processing model. Language processing model may include a program automatically generated by computing device and/or language processing module to produce associations between one or more words extracted from at least a document and detect associations, including without limitation mathematical associations, between such words. Associations between language elements, where language elements include for purposes herein extracted words, relationships of such categories to other such term may include, without limitation, mathematical associations, including without limitation statistical correlations between any language element and any other language element and/or language elements. Statistical correlations and/or mathematical associations may include probabilistic formulas or relationships indicating, for instance, a likelihood that a given extracted word indicates a given category of semantic meaning. As a further example, statistical correlations and/or mathematical associations may include probabilistic formulas or relationships indicating a positive and/or negative association between at least an extracted word and/or a given semantic meaning; positive or negative indication may include an indication that a given document is or is not indicating a category semantic meaning. Whether a phrase, sentence, word, or other textual element in a document or corpus of documents constitutes a positive or negative indicator may be determined, in an embodiment, by mathematical associations between detected words, comparisons to phrases and/or words indicating positive and/or negative indicators that are stored in memory at computing device, or the like.

Still referring to 1, language processing module and/or diagnostic engine may generate the language processing model by any suitable method, including without limitation a natural language processing classification algorithm; language processing model may include a natural language process classification model that enumerates and/or derives statistical relationships between input terms and output terms. Algorithm to generate language processing model may include a stochastic gradient descent algorithm, which may include a method that iteratively optimizes an objective function, such as an objective function representing a statistical estimation of relationships between terms, including relationships between input terms and output terms, in the form of a sum of relationships to be estimated. In an alternative or additional approach, sequential tokens may be modeled as chains, serving as the observations in a Hidden Markov Model (HMM). HMMs as used herein are statistical models with inference algorithms that that may be applied to the models. In such models, a hidden state to be estimated may include an association between an extracted words, phrases, and/or other semantic units. There may be a finite number of categories to which an extracted word may pertain; an HMM inference algorithm, such as the forward-backward algorithm or the Viterbi algorithm, may be used to estimate the most likely discrete state given a word or sequence of words. Language processing module may combine two or more approaches. For instance, and without limitation, machine-learning program may use a combination of Naive-Bayes (NB), Stochastic Gradient Descent (SGD), and parameter grid-searching classification techniques; the result may include a classification algorithm that returns ranked associations.

Continuing to refer to FIG. 1, generating language processing model may include generating a vector space, which may be a collection of vectors, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each vector in an n-dimensional vector space may be represented by an n-tuple of numerical values. Each unique extracted word and/or language element as described above may be represented by a vector of the vector space. In an embodiment, each unique extracted and/or other language element may be represented by a dimension of vector space; as a non-limiting example, each element of a vector may include a number representing an enumeration of co-occurrences of the word and/or language element represented by the vector with another word and/or language element. Vectors may be normalized, scaled according to relative frequencies of appearance and/or file sizes. In an embodiment associating language elements to one another as described above may include computing a degree of vector similarity between a vector representing each language element and a vector representing another language element; vector similarity may be measured according to any norm for proximity and/or similarity of two vectors, including without limitation cosine similarity, which measures the similarity of two vectors by evaluating the cosine of the angle between the vectors, which can be computed using a dot product of the two vectors divided by the lengths of the two vectors. Degree of similarity may include any other geometric measure of distance between vectors.

Still referring to FIG. 1, language processing module may use a corpus of documents to generate associations between language elements in a language processing module, and diagnostic engine may then use such associations to analyze words extracted from one or more documents and determine that the one or more documents indicate significance of a category. In an embodiment, language module and/or computing device 104 may perform this analysis using a selected set of significant documents, such as documents identified by one or more experts as representing good information; experts may identify or enter such documents via graphical user interface, or may communicate identities of significant documents according to any other suitable method of electronic communication, or by providing such identity to other persons who may enter such identifications into computing device 104. Documents may be entered into a computing device by being uploaded by an expert or other persons using, without limitation, file transfer protocol (FTP) or other suitable methods for transmission and/or upload of documents; alternatively or additionally, where a document is identified by a citation, a uniform resource identifier (URI), uniform resource locator (URL) or other datum permitting unambiguous identification of the document, diagnostic engine may automatically obtain the document using such an identifier, for instance by submitting a request to a database or compendium of documents such as JSTOR as provided by Ithaka Harbors, Inc. of New York.

As a non-limiting example, risk calculation module may factor in data from various genetic markers related to Alzheimer's disease, cardiovascular disease, metabolism, and other relevant factors. By analyzing these markers collectively, the module may generate PRS that quantifies the individual's risk level, enabling healthcare practitioners to make informed decisions regarding preventive strategies and clinical management approaches. In an embodiment, wherein calculating PRS 148 further comprise aggregating a genetic risk factor 164. As used in this disclosure, a “genetic risk factor” refers to specific genetic markers, variations, or traits that have been identified through scientific research and studies as contributing to the susceptibility or predisposition of an individual to a particular health condition. Genetic risk factors may encompass a wide range of specific genetic markers associated with various aspects of neurodegenerative diseases, including factors like the ApoE gene alleles, as well as other relevant genetic variations linked to disease susceptibility. The aggregation process within risk calculation module may integrate the influence of these genetic risk factors by considering the individual effect sizes, reported associations with disease risk, and significance. In another embodiment, wherein calculating PRS may further comprise multiplying PRS by each SNP's reported effect size. Within risk calculation module, multiplication operation involves assigning a weight to each SNP based on its established impact on disease risk, as indicated by reported effect size from previous research and studies. To determine these weights may involve a multi-step process that draws from existing data. To obtain information on the impact of each SNP from extensive databases, research studies, and scientific literature that have established associations between specific SNPs and disease risk. These associations may be derived from large-scale genetic studies and analyses that have identified genetic variations linked to various health conditions. The weights assigned to each SNP may not be predetermined or fixed, rather, they may be calculated based on the reported effect size of each SNP. Effect size may represent the magnitude of the relationship between a genetic variant and the associated disease risk. Larger effect sizes may indicate a stronger impact on disease risk, while smaller effect size may indicate a more modest influence. In some embodiments, weights may be generated using a machine-learning module, such as weight machine-learning module disclosed above. In some embodiments, weights may be generated using language processing module as disclosed above. By multiplying PRS by effect sizes, risk calculation module may account for the varying contributions of different SNPs to an individual's overall risk profile. SNPs with larger effect sizes, signifying a stronger association with disease risk, receive a higher weight in the calculation. Conversely, SNPs with smaller effect sizes have a comparatively smaller impact on the final PRS. In a further embodiment, the process of calculating PRS involves an additional step of weighing the multiplied PRS, may be obtained by multiplying the PRS by each SNP reported effect size, against population-based incidence reports. Population-based incidence reports serve as a reference for longitudinal prediction, allowing for a more accurate estimation of an individual's risk for neurodegenerative diseases over time. Within risk calculation module, weighing process may consider the interplay between the individual's genetic makeup, as represented by PRS, and the incidence rates of neurodegenerative diseases observed in broader populations. By aligning PRS with population-based data, risk calculation module may provide a contextualized risk assessment that takes into account the prevalence of these diseases in the general population. As a non-limiting example, a user who may undergoes genetic testing to assess risk for neurodegenerative diseases. Risk calculation module aggregates genetic risk factors associated with ApoE gene alleles, other relevant SNPs, and genetic markers. These factors may be assigned individual weights based on reported effect sizes, reflecting their influence on disease risk. For instance, an SNP linked to a substantial increase in risk receives a higher weight than an SNP with a more moderate effect size. After obtaining the weighted contributions from each genetic risk factor, Risk calculation module may generate a multiplied PRS for the user. This multiplied PRS is then weighed against population-based incidence reports. Suppose that population data may indicate a moderate prevalence of neurodegenerative diseases among individuals with similar genetic profiles. By aligning user's multiplied PRS with this population-based context, risk calculation module may provide a risk assessment that factors in both genetic predisposition and the broader epidemiological context. As a result, user's PRS may reflect their personalized risk for neurodegenerative diseases, incorporating the combined impact of various genetic risk factors, their reported effect sizes, and the prevalence of these diseases in the population.

With continued reference to FIG. 1, calculating polygenic risk score (PRS) 148 further comprises identifying the mitochondrial haplotype and SNPs. The mitochondrial haplotype may signify the specific genetic variants and markers present in an individual's mitochondrial DNA. Alongside the mitochondrial haplotype, PRS also encompasses the identification of SNPs, which are variations in a single nucleotide within the DNA sequence. As a non-limiting example, PRS may be assigned to a user may not only be based on the known genetic markers linked to these diseases but also includes the identification of their mitochondrial haplotype and specific SNPs. The mitochondrial haplotype information may provide insights into user's maternal lineage and genetic traits inherited through the maternal line. This is relevant because mitochondrial DNA is inherited exclusively from the mother and may carry important insights into an individual's genetic heritage. Additionally, PRS takes into account specific SNPs present in user's genome. These SNPs, distributed across various genes, contribute to the overall genetic makeup that influences health and disease susceptibility.

With continued reference to FIG. 1, mitochondrial haplotype and SNPs may further comprise identifying specific genetic markers associated with AD risk gene, AD risk factors (e.g., cardiovascular disease (CVD), metabolic health, periodontal disease, depression, and the like), and relevant pharmacogenomic markers (e.g., lipoprotein (a) and proprotein convertase subtilisin/kexin type 9 serine protease [PCSK9] variants, and the like). This analysis may involve a multi-step process aimed at uncovering genetic variation that may contribute to an individual's susceptibility to neurodegenerative diseases. The process may begin with the extraction and assessment of genetic data from the user genome, which may include targeted regions associated with AD risk gene and other relevant factors. Next, specialized algorithms and data processing techniques may be employed to identify specific genetic markers within these regions. Furthermore, genetic markers may influence cardiovascular health, metabolic functions, and other risk factors are scrutinized to ascertain their presence or absence. This examination may involve comparing the user genetic makeup against established databases and reference genomes, allowing for the identification of significant genetic variations associated with disease risk. Additionally, pharmacogenomic markers, such as those related to lipid metabolism or drug response, may be analyzed to understand how these variations may interact with an individual's genetic profile and contribute to their susceptibility to neurodegenerative diseases. As a non-limiting example, the analysis of mitochondrial haplotype and SNPs may reveal variations in genes such as ApoE that may be linked to increased risk of Alzheimer's disease, genetic markers influencing cardiovascular health, and variants affecting lipid metabolism, may provide a comprehensive genetic landscape that contributes to the individual's overall neurodegenerative disease risk profile.

Still referring to FIG. 1, apparatus 100 is configured to display a personalized risk profile 168 through a display device 172. As used in this disclosure, a “display device” is any device capable of rendering visual content. This may include, as non-limiting examples, a computer monitor, a mobile device screen, or a projection device. This display provides individuals with a clear and comprehensible representation of their personalized risk assessment for neurodegenerative diseases, enabling them to make informed decisions about their health and engage in proactive management strategies. As a non-limiting example, access their personalized risk profile 168 by simply logging into a web-based platform via their computer or mobile device. Once logged in, display device 172, which could be their computer monitor or smartphone screen, presents a user-friendly interface showing their risk scores, genetic markers, and relevant information about their susceptibility to neurodegenerative diseases. This visual representation empowers users to understand their potential health risks and encourages them to explore recommendations for lifestyle modifications, medical interventions, or regular screenings based on their risk levels.

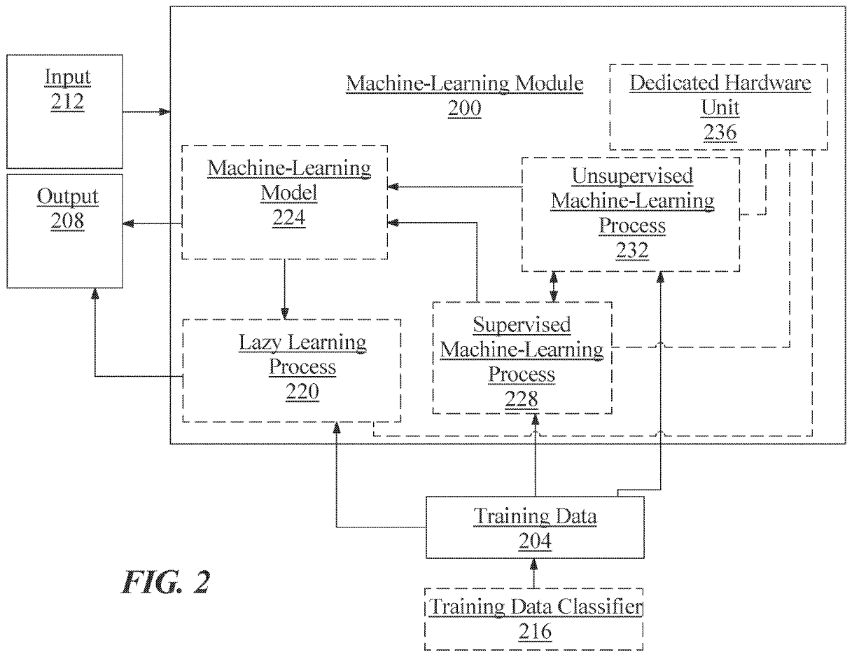

Referring now to FIG. 2, an exemplary embodiment of a machine-learning module 200 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 204 to generate an algorithm instantiated in hardware or software logic, data structures, and/or functions that will be performed by a computing device/module to produce outputs 208 given data provided as inputs 212; this is in contrast to a non-machine learning software program where the commands to be executed are determined in advance by a user and written in a programming language.

Still referring to FIG. 2, “training data,” as used herein, is data containing correlations that a machine-learning process may use to model relationships between two or more categories of data elements. For instance, and without limitation, training data 204 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 204 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 204 according to various correlations; correlations may indicate causative and/or predictive links between categories of data elements, which may be modeled as relationships such as mathematical relationships by machine-learning processes as described in further detail below. Training data 204 may be formatted and/or organized by categories of data elements, for instance by associating data elements with one or more descriptors corresponding to categories of data elements. As a non-limiting example, training data 204 may include data entered in standardized forms by persons or processes, such that entry of a given data element in a given field in a form may be mapped to one or more descriptors of categories. Elements in training data 204 may be linked to descriptors of categories by tags, tokens, or other data elements; for instance, and without limitation, training data 204 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 2, training data 204 may include one or more elements that are not categorized; that is, training data 204 may not be formatted or contain descriptors for some elements of data. Machine-learning algorithms and/or other processes may sort training data 204 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 204 to be made applicable for two or more distinct machine-learning algorithms as described in further detail below. Training data 204 used by machine-learning module 200 may correlate any input data as described in this disclosure to any output data as described in this disclosure. As a non-limiting illustrative the training data could consist of input variables like individual genetic variants identified from whole genome sequencing (WGS), mitochondrial haplogroup information, demographic details, cholesterol levels, blood pressure measurements, and user-reported lifestyle factors such as exercise frequency and dietary habits. These inputs are then correlated with the corresponding output data, which includes calculated polygenic risk scores (PRS) for neurodegenerative diseases and the associated risk levels. The machine-learning module learns to recognize patterns and relationships within the training data, allowing it to generate accurate and personalized risk assessments based on similar input data provided by new users.

Further referring to FIG. 2, training data may be filtered, sorted, and/or selected using one or more supervised and/or unsupervised machine-learning processes and/or models as described in further detail below; such models may include without limitation a training data classifier 216. Training data classifier 216 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 200 may generate a classifier using a classification algorithm, defined as a processes whereby a computing device and/or any module and/or component operating thereon derives a classifier from training data 204. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 216 may classify elements of training data to segment training data into cohorts defined by certain genetic variants associated with Alzheimer's disease, such as the ApoE gene alleles, to focus on individuals with elevated risk profiles. Alternatively, it could categorize training data based on demographic criteria like age, gender, or family history to create cohorts representing distinct risk strata.

With further reference to FIG. 2, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Still referring to FIG. 2, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value.

As a non-limiting example, and with further reference to FIG. 2, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

With continued reference to FIG. 1, at least a epigenetic analysis module 152 is configured to process a genome. As used in this disclosure, an “epigenetic analysis module” refers to a component within apparatus that is configured to analyze gene expression. The epigenetic analysis module 152 may analyze epigenetic modifications in an individual's genome, focusing on modifications that may influence gene expression patterns without altering the underlying DNA sequence. Epigenetic analysis module 152 may delve into epigenetic marks such as DNA methylation, histone modifications, the module may gain insights into how environmental factors, lifestyle, and genetic predispositions interact to impact user's health and disease susceptibility. Epigenetic analysis module 152 may be designed to analyze the epigenetic information present within a genome. Epigenetic modifications may include inheritable changes that affect gene expression without altering the underlying DNA sequence. Epigenetic analysis module 152 may employ advanced computational algorithms to decipher and interpret modifications, may aim to identify patterns and correlations between epigenetic marks and disease-related outcomes. The epigenetic analysis module 152, may be equipped with advanced machine-learning capabilities, may be configured to examine an individual's genome for specific epigenetic patterns. Diverse epigenetic datasets may encompass a wide range of known epigenetic modifications, allowing the module to identify subtle variations and deviations from established patterns. Rather than relying on a fixed list of patterns, the module's may adaptive nature enables it to detect epigenetic signatures associated with different health conditions. Within this training process, epigenetic analysis module 152 may learn to recognize and interpret these marks in the context of disease risk assessment. Epigenetic analysis module 152 may contribute to a more comprehensive understanding of their personalized risk profile for neurodegenerative diseases. As a non-limiting example, epigenetic analysis module 152 may detect differential DNA methylation patterns in certain genomic regions associated with genes involved in inflammation, a known risk factor for neurodegenerative diseases. By recognizing such patterns, epigenetic analysis module 152 may contribute valuable insights into the user risk profile.

Continuing to refer to FIG. 2, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 2, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Still referring to FIG. 2, machine-learning module 200 may be configured to perform a lazy-learning process 220 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 204. Heuristic may include selecting some number of highest-ranking associations and/or training data 204 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 2, machine-learning processes as described in this disclosure may be used to generate machine-learning models 224. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 224 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 224 may be generated by creating an artificial neural network, such as a convolutional neural network comprising an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 2, machine-learning algorithms may include at least a supervised machine-learning process 228. At least a supervised machine-learning process 228, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include input as described in this disclosure as input, outputs as described in this disclosure as output and a scoring function representing a desired form of relationship to be detected between inputs and outputs: scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 204. Persons skilled in the art, upon reviewing the entirety of this disclosure, will be aware of various possible variations of at least a supervised machine-learning process 228 that may be used to determine relation between inputs and outputs. Supervised machine-learning processes may include classification algorithms as defined above.

With further reference to FIG. 2, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 2, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 2, machine learning processes may include at least an unsupervised machine-learning processes 232. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 232 may not require a response variable; unsupervised processes 232 may be used to find interesting patterns and/or inferences between variables, to determine a degree of correlation between two or more variables, or the like.

Still referring to FIG. 2, machine-learning module 200 may be designed and configured to create a machine-learning model 224 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 2, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 2, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 2, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 2, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 2, one or more processes or algorithms described above may be performed by at least a dedicated hardware unit 236. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 236 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 236 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 236 to perform one or more operations described herein, such as evaluation of model and/or algorithm outputs, one-time or iterative updates to parameters, coefficients, weights, and/or biases, and/or any other operations such as vector and/or matrix operations as described in this disclosure.

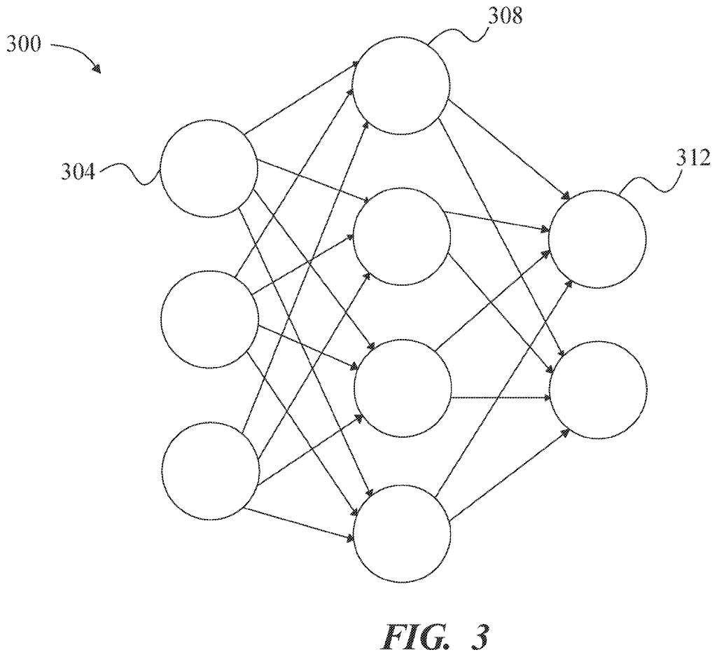

Referring now to FIG. 3, an exemplary embodiment of neural network 300 is illustrated. A neural network 300 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 304, one or more intermediate layers 308, and an output layer of nodes 312. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

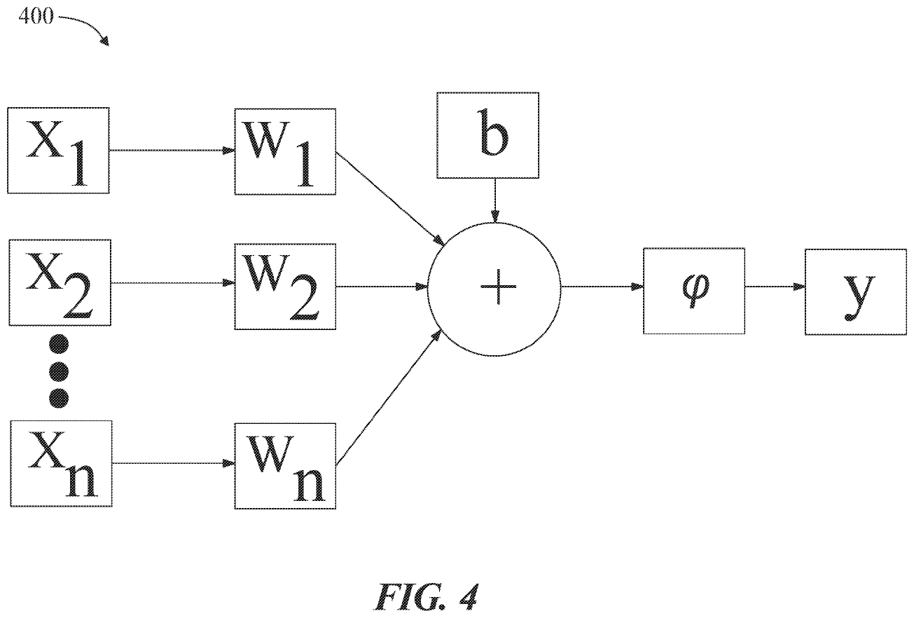

Referring now to FIG. 4, an exemplary embodiment of a node 400 of a neural network is illustrated. A node may include, without limitation a plurality of inputs xᵢ that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as f(x)=tanh²(x), a rectified linear unit function such as f(x)=max(0, x), a “leaky” and/or “parametric” rectified linear unit function such as f(x)=max(ax, x) for some a, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as f(x)=x*sigmoid(x), a Gaussian error linear unit function such as f(x)=a(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights w/may be determined by training a neural network using training data, which may be performed using any suitable process as described above.

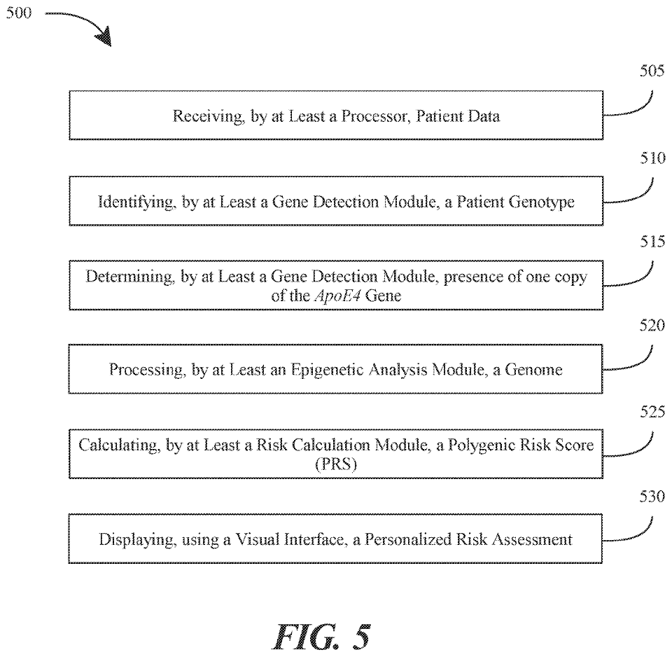

Referring now to FIG. 5, a flow diagram of an exemplary method 500 for generating a personalized risk assessment for neurodegenerative disease is illustrated. Method 500 includes a step 505 of receiving, by at least a processor, user data, wherein the user data comprises user genetic data. This may be implemented, without limitation, as described above with reference to FIGS. 1-4.

With continued reference to FIG. 5, method 500 includes a step 510 of identifying, by at least a gene detection module of the at least a processor, wherein the at least a genotype identification module is configured to identify a user genotype. This may be implemented, without limitation, as described above with reference to FIGS. 1-4.

With continued reference to FIG. 5, method 500 includes a step 515 of determining, by at least a gene detection module of the at least a processor, wherein the at least a gene detection module comprises identifying the presence of one copy of the ApoE4 gene. This may be implemented, without limitation, as described above with reference to FIGS. 1-4.

With continued reference to FIG. 5, method 500 includes a step 520 of processing, by at least an epigenetic analysis module 152, wherein the at least a epigenetic analysis module 152 is configured to process a genome. This may be implemented, without limitation, as described above with reference to FIGS. 1-4.

With continued reference to FIG. 5, method 500 includes a step 525 of calculating, using at least a risk calculation module, a polygenic risk score (PRS). This may be implemented, without limitation, as described above with reference to FIGS. 1-4.

With continued reference to FIG. 5, method 500 includes a step 530 of displaying, by at least a processor, the personalized risk assessment using a visual interface at a display device. This may be implemented, without limitation, as described above with reference to FIGS. 1-4.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

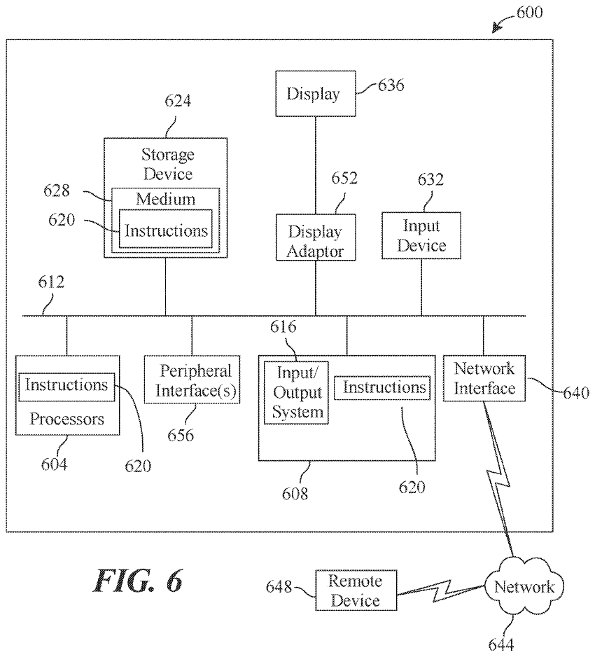

FIG. 6 shows a diagrammatic representation of one embodiment of a computing device in the exemplary form of a computer system 600 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 600 includes a processor 604 and a memory 608 that communicate with each other, and with other components, via a bus 612. Bus 612 may include any of several types of bus structures including, but not limited to, a memory bus, a memory controller, a peripheral bus, a local bus, and any combinations thereof, using any of a variety of bus architectures.

Processor 604 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 604 may be organized according to Von Neumann and/or Harvard architecture as a non-limiting example. Processor 604 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 608 may include various components (e.g., machine-readable media) including, but not limited to, a random-access memory component, a read only component, and any combinations thereof. In one example, a basic input/output system 616 (BIOS), including basic routines that help to transfer information between elements within computer system 600, such as during start-up, may be stored in memory 608. Memory 608 may also include (e.g., stored on one or more machine-readable media) instructions (e.g., software) 620 embodying any one or more of the aspects and/or methodologies of the present disclosure. In another example, memory 608 may further include any number of program modules including, but not limited to, an operating system, one or more application programs, other program modules, program data, and any combinations thereof.

Computer system 600 may also include an input device 632. In one example, a user of computer system 600 may enter commands and/or other information into computer system 600 via input device 632. Examples of an input device 632 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 632 may be interfaced to bus 612 via any of a variety of interfaces (not shown) including, but not limited to, a serial interface, a parallel interface, a game port, a USB interface, a FIREWIRE interface, a direct interface to bus 612, and any combinations thereof. Input device 632 may include a touch screen interface that may be a part of or separate from display 636, discussed further below. Input device 632 may be utilized as a user selection device for selecting one or more graphical representations in a graphical interface as described above.

Computer system 600 may also include a storage device 624. Examples of a storage device (e.g., storage device 624) include, but are not limited to, a hard disk drive, a magnetic disk drive, an optical disc drive in combination with an optical medium, a solid-state memory device, and any combinations thereof. Storage device 624 may be connected to bus 612 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 624 (or one or more components thereof) may be removably interfaced with computer system 600 (e.g., via an external port connector (not shown)). Particularly, storage device 624 and an associated machine-readable medium 628 may provide nonvolatile and/or volatile storage of machine-readable instructions, data structures, program modules, and/or other data for computer system 600. In one example, software 620 may reside, completely or partially, within machine-readable medium 628. In another example, software 620 may reside, completely or partially, within processor 604.

A user may also input commands and/or other information to computer system 600 via storage device 624 (e.g., a removable disk drive, a flash drive, etc.) and/or network interface device 640. A network interface device, such as network interface device 640, may be utilized for connecting computer system 600 to one or more of a variety of networks, such as network 644, and one or more remote devices 648 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 644, may employ a wired and/or a wireless mode of communication. In general, any network topology may be used. Information (e.g., data, software 620, etc.) may be communicated to and/or from computer system 600 via network interface device 640.

Computer system 600 may further include a video display adapter 652 for communicating a displayable image to a display device, such as display device 636. Examples of a display device include, but are not limited to, a liquid crystal display (LCD), a cathode ray tube (CRT), a plasma display, a light emitting diode (LED) display, and any combinations thereof. Display adapter 652 and display device 636 may be utilized in combination with processor 604 to provide graphical representations of aspects of the present disclosure. In addition to a display device, computer system 600 may include one or more other peripheral output devices including, but not limited to, an audio speaker, a printer, and any combinations thereof. Such peripheral output devices may be connected to bus 612 via a peripheral interface 656. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

It is to be noted that any one or more of the aspects and embodiments described herein may be conveniently implemented using digital electronic circuitry, integrated circuitry, specially designed application specific integrated circuits (ASICs), field programmable gate arrays (FPGAs) computer hardware, firmware, software, and/or combinations thereof, as realized and/or implemented in one or more machines (e.g., one or more computing devices that are utilized as a user computing device for an electronic document, one or more server devices, such as a document server, etc.) programmed according to the teachings of the present specification, as will be apparent to those of ordinary skill in the computer art. These various aspects or features may include implementation in one or more computer programs and/or software that are executable and/or interpretable on a programmable system including at least one programmable processor, which may be special or general purpose, coupled to receive data and instructions from, and to transmit data and instructions to, a storage system, at least one input device, and at least one output device. Appropriate software coding may readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations may be within the scope of the following claims.

1. An apparatus for generating a personalized risk assessment for neurodegenerative disease, the apparatus comprising:
at least a processor; and
a memory communicatively connected to the at least a processor, wherein the memory contains instruction configuring the at least a processor to:
receive user data pertaining to a user, wherein the user data comprises user genetic data;
process the user genetic data of the user, wherein processing the user genetic data comprises:
generating a genotype identification module using the user genetic data, wherein the genotype identification module is configured to identify a user genotype;
generating a gene detection module using the user genetic data, wherein the gene detection module is configured to:
determine the presence of one copy of an apolipoprotein (ApoE)4 gene; and
identify an upstream and downstream regulatory region of ApoE4 and translocase of outer mitochondrial membrane 40 (TOMM40) gene to assess potential impact on disease risk;
examine the user;
generate a genetic profile, wherein the genetic profile comprises a personalized risk profile; and
display the personalized risk profile through a display device.

2. The apparatus of claim 1, the neurodegenerative disease further comprises Alzheimer's disease.

3. The apparatus of claim 1, wherein the personalized risk profile is based on genetic factors associated with the ApoE gene.

4. The apparatus of claim 1, wherein the user genetic data further comprises:
direct-to-consumer genetic data; and
traditional biological data.

5. The apparatus of claim 1, wherein processing the user genetic data, the genotype identification module is configured to identify user's ApoE genotype.

6. The apparatus of claim 1, wherein generating the gene detection module further comprises identifying the upstream and downstream regulatory region of ApoE and TOMM40.

7. The apparatus of claim 1, wherein processing the user genetic data comprises using a haplogroup examination module configured to examine the user mitochondrial haplogroup.

8. The apparatus of claim 7, wherein using the haplogroup examination module further comprises:
analyzing specific genetic markers associated with mitochondrial DNA; and
determining ancestral lineage and genetic characteristics of the user's mitochondrial genome.

9. The apparatus of claim 1, further comprising a risk calculation module, wherein the risk calculation module is configured to:
identifying the user's risk for onset of the neurodegenerative disease based on analysis of genomic information, traditional biological data, and specific genetic risk factors;
integrating a reports effected size of individual Single Nucleotide Polymorphisms (SNP); and
weighing the impacts of individual variants against population-based incident reports.

10. The apparatus of claim 9, within the risk calculation module is further configured to aggregate data from various genetic markers, including APOE gene alleles.

11. A method for generating a personalized risk assessment for neurodegenerative disease, the method comprising:
receiving, by at least a processor, user data, wherein the user data comprises a user genetic data;
identifying, by at least a gene detection module of the at least a processor, wherein the at least a genotype identification module is configured to identify a user genotype;
determining, by at least a gene detection module of the at least a processor, wherein the at least a gene detection module comprises identifying a presence of one copy of a ApoE4 variant;
processing, by at least an epigenetic analysis module, wherein the at least an epigenetic analysis module is configured to process a genome; and
calculating, using at least a risk calculation module, a polygenic risk score (PRS).

12. The method of claim 11, wherein receiving the user data comprises at-home blood test data.

13. The method of claim 11, wherein receiving the user data further comprises interpreting genetic information in the context of the individual's predisposing conditions, family history, lifestyle, and serum biomarkers.

14. The method of claim 11, wherein receiving the genetic user data further comprises receiving direct-to-consumer genomic information and traditional biological data of a user.

15. The method of claim 11, wherein analyzing a genetic risk factor further comprises analyzing risk factors for Alzheimer's disease, neurodegenerative disease, cardiovascular disease, inflammatory disease, and metabolism.

16. The method of claim 11, wherein processing the genome comprises conducting whole genome sequencing (WGS).

17. The method of claim 11, wherein processing the genome further comprises determining a whole exome sequence.

18. The method of claim 11, wherein the calculating, using at least a risk calculation module, the PRS further comprises identifying the mitochondrial haplotype and SNPs.

19. The method of claim 18, wherein the step of identifying the mitochondrial haplotype and SNPs further comprises identifying specific genetic markers associated with:
Alzheimer's disease risk genes;
Alzheimer's disease risk factors; and
relevant pharmacogenomic markers.

20. The method of claim 11, wherein calculating the polygenic risk scores (PRS), the method further comprises:
aggregating a genetic risk factor,
multiplying PRS by each SNP's reported effect size; and
weighing the multiplied PRS by SNP result against population-based incidence reports for longitudinal prediction.
